(19) United States
(12) Patent Application Publication
(10) Pub. No.: US 2009/0326259 A1
(43) Pub. Date: Dec. 31, 2009

(54) NF-Kappabeta Activation Inhibitor

(75) Inventors: Hideki Ushio, Minato-ku (JP); Reiko Nagasaka, Wako-shi (JP); Kazuyuki Ohhara, Ohta-ku (JP); Hiroshi Ozaki, Bunkyo-ku (JP); Masatoshi Hori, Bunkyo-ku (JP)

Correspondence Address: OLIFF & BERRIDGE, PLC, P.O. BOX 320850, ALEXANDRIA, VA, 22320-4850, US

(73) Assignees: Tokyo University of Marine Science and Technology, Tokyo (JP); The University of Tokyo, Tokyo (JP)

(21) Appl. No.: 12/308,364

(22) PCT Filed: Jun. 13, 2007
(86) PCT No.: PCT/JP2007/061917

§ 371 Date: Dec. 12, 2008

(30) Foreign Application Priority Data
Jun. 14, 2006 (JP) 2006-165308

(51) Int. Cl. C07C 69/76

(52) U.S. Cl. 560/104

(57) ABSTRACT

The object is to provide a highly safe NF-κB activation inhibitor at a low cost which can be used for prevention and treatment of diseases associated with the activation of NF-κB such as inflammation or type-2 diabetes. Specifically, the NF-κB activation inhibitor comprises a compound represented by the chemical structural formula depicted in the following Chemical Formula 1 [wherein R₁ represents a hydroxy group; R₂ represents a hydroxy group, a methoxy group or an alkoxy group; and R₃ represents an ester comprising triterpene or a salt thereof] or a salt of the compound as active ingredient. A representative example of the compound is hydroxycinnamic acid derivative triterpene alcohol ester.

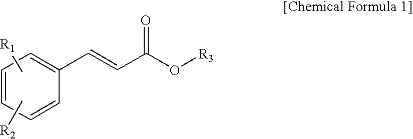

### 1. INTRODUCTION

The present invention relates to a NF-κB activation inhibitor, which can be used for prevention or treatment of diseases such as inflammation or type-2 diabetes.

### 2. BACKGROUND OF THE INVENTION

Control of gene expression can be achieved by the binding of a transcription regulator (a DNA binding protein) to a specific DNA response sequence. Nuclear Factor kappa B (NF-κB), as a known transcription regulator, had been considered as a B cell-specific factor conjugating with the enhancer for kappa light-chain of immunoglobulin. Later, as a major transcription regulator, NF-κB was discovered to have important functions of, among others, controlling the expression of genes and thus controlling immune response or inflammation through binding to the promoters for cytokines or receptors involved in all kinds of inflammation. Therefore NF-κB plays an important causative role in disease symptoms of immune response or inflammation. Compounds capable of inhibiting NF-κB activation can be of use in improving and treating these diseases.

Consequently, further exploration of inhibiting agents of NF-κB activation reports that non-steroid anti-inflammatory of aspirin or sodium salicylate at a higher concentration takes effect in inhibiting NF-κB activation, purin compound of xanthine derivative and steroid compound of dexamethasone trigger inhibition of NF-κB activation, and turmeric ingredient of curcumin and propolis ingredient of cinnamic acid phenethyl ester demonstrate inhibition of NF-κB activation.

Activated NF-κB was found as well inhibiting activation of peroxisome proliferator-activated receptor (PPARγ), a nuclear receptor-type transcription regulator, which controls the induction and differentiation of adipocytes and mediates the overall lipid metabolism.

However, the NF-κB activation inhibitor concerned in the present invention comprises a compound represented by the chemical structural formula depicted in the following Chemical Formula 1, which compound is a basic structure of an ester bound with triterpene alcohol by propionic acid derivative with a phenyl group.

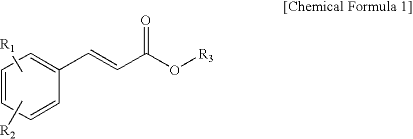

The following describes prior art of the compound from the perspective of chemical structure. In Chemical Formula 1, R₁ represents a hydroxy group, R₂ represents a methoxy group, and R₃ represents a triterpene compound, which three are collectively referred to as γ-oryzanol. U.S. Pat. No. 3,493,459 discloses sebum secretion of the compound, though, without dealing with its mechanism and records no NF-κB activation inhibition advanced in this invention.

Akihisa et al. report in the non-patent literature 1 below that ingredients of γ-oryzanol demonstrate anti-inflammatory effect in murine auricular inflammation caused by phorbol ester, but note no NF-κB activation inhibition therein.

A free ferulic acid derivative with R₁ representing hydroxy group, R₂ methoxy group and R₃ hydrogen is recorded as an antioxidant in the below-mentioned non-patent literature 2, which mentions no inhibitory effect of NF-κB activation as well.

The compound of the present invention is among compounds widely distributed in edible plant kingdom, whose safety has been confirmed in long-term consumption so far. Nevertheless, its inhibition of NF-κB activation has never been noticed.
Patent literature 1: Official Gazette No. 3493459 for Japanese patent
Patent literature 2: Tokuhyou Gazette No. 2005-501043 for Japanese patent
Non-patent literature 1: Akihisa, T., Yamaura, K., Ukiya, M., Kimura, Y., Shimizu, N., Arai, K. Triterpene alcohol and sterol ferulates from rice bran and their anti-inflammatory effects, J. Agric. Food Chem., 48, 2313-2319 (2000)
Non-patent literature 2: Rice-Evans, C. A., Miller, N. J, Paganga, G Structure-antioxidant activity relationships of flavonoids and phenolic acids. Free Radical Biology & Medicine, 20, 933-956 (1996).

### 3. SUMMARY OF THE INVENTION

The present invention aims at a highly safe NF-κB activation inhibitor, which can be used for prevention or treatment of diseases such as inflammation or type-2 diabetes.

The NF-κB activation inhibitor according to this invention comprises a compound represented by the following Chemical Formula 1 or a salt thereof as active ingredient.

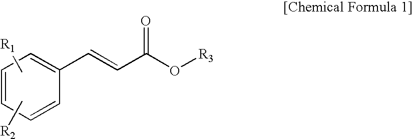

In the chemical structural formula depicted in Chemical Formula 1, R₁ represents a hydroxy group; R₂ represents a hydroxy group, a methoxy group or an alkoxy group; and R₃ represents a backbone of triterpene such as sterol.

Representative compounds of Chemical Formula 1 can be hydroxycinnamic acid derivative triterpene alcohol ester, select-grade cycloartenyl ferulate, β-sitosteryl ferulate, stigmasteryl ferulate, 24-methylene cycloartenyl ferulate or campesteryl ferulate, or a salt thereof. The salt is among approved pharmaceutical select-grade salts, like ammonium, for example.

Administration of the inhibitor of NF-κB activation according to the present invention may adopt known methods such as non-oral or oral routes. When given in non-oral forms, the formulations are not restricted and may be any of the following: intravenous injection (including intravenous drip), intramuscular injection, intraperitoneal injection, subcutaneous injection, nasal drop, suppository, ointment, cream, unction and etc.; When given in oral forms, the formulations are not restricted and may be any of the following: tablet, capsule, granule, powder, pellet, troche, aqueous solution for oral intake, suspension, emulsion, syrup and etc., or a drier which needs to be dissolved for use.

These oral or non-oral forms with a variety of usages, which are prepared through routine methods, may select to add appropriate adjuvants commonly used in the manufacture of pharmaceutical products, including excipient, bulk additive, extender, adhesive, wetting agent, disintegrant, lubricant, surfactant, disperser, buffer, preservative, cosolvent, antiseptic, deodorizer, analgesic, stabilizer and osmoregulator, and the like. These forms may be applied in cosmetic raw materials, food raw materials, beverages, livestock feed and pet foods.

The compound concerned in this invention inhibits the activation of NF-κB and may be employed to prevent and treat NF-κB activation-related large-area inflammatory diseases, such as ulcerative colitis and enteritis (including Crohn's disease), or adiponectin-related diseases, such as insulin-resistant type-2 diabetes.

### 4. DESCRIPTION OF THE FIGURES

FIG. 1. Diagram for isolate example of hydroxycinnamic acid derivative triterpene alcohol ester.

FIG. 2 Diagram for effect of cycloartenyl ferulate upon NF-κB activation in RAW264.7 macrophage.

FIG. 3 Diagram for effect of cycloartenyl ferulate (CAF) upon iNOS mRNA expression in RAW264.7 macrophage.

FIG. 4 Diagram for effect of cycloartenyl ferulate (CAF) upon IL-1β mRNA expression in RAW264.7 macrophage.

FIG. 5 Diagram for effect of γ-oryzanol upon DAI score in DSS enteritis.

FIG. 6 Diagram for effect of γ-oryzanol upon MPO activity in DSS enteritis.

FIG. 7 Microscopic view of HE-stained murine colon.

FIG. 8 Diagram for effect of NF-κB activation inhibition upon the secretion of adiponectin in murine adipocytes.

### 5. PREFERRED EMBODIMENTS

With the adoption of ingredients in rice bran whose safety has been confirmed by long-term consumption experience, the goal of preventing or treating diseases like inflammation or type-2 diabetes has been achieved through inhibition of NF-κB activation.

Below are the preferred embodiments of the present invention, which are provided only for better comprehension of the invention, without limiting the scope thereof.

### Embodiment 1

[Extraction example of hydroxycinnamic acid derivative triterpene alcohol ester] Chloroform methanol mixture is used to extract whole lipids from rice bran. The resulting extract solvent is displaced in a 94:2:6 (v/v) mixture of acetonitrile (Kokusan Chemical):acetic acid (Kokusan Chemical):distilled water (Kokusan Chemical) and is filtered with a 0.50 μm mini-filter (PTFE, ADVANTEC TOYO).

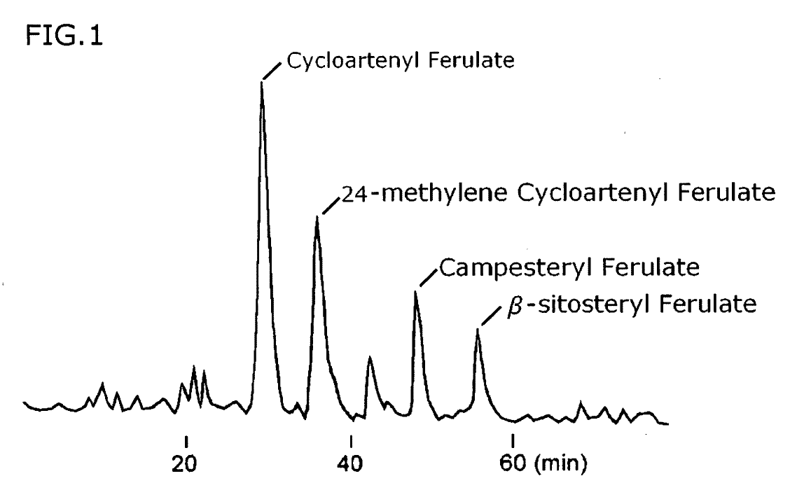

The 94:2:6 (v/v) mixture of acetonitrile (Kokusan Chemical):acetic acid (Kokusan Chemical):distilled water (Kokusan Chemical) is chosen as the mobile phase and the reversed phase HPLC column Mightysil RP-18 GP250-4.63 μm (Kanto Chemical) as the stationary phase, both of which are subject to separation at a flow rate of 1 ml/min in high performance liquid chromatographic system. Using RF-10A (Shimadzu Co.) to detect at an excitation wavelength of 330 nm and at fluorescence intensity with fluorescence emission wavelength of 390 nm ends with what's shown in FIG. 1.

Based upon the result as shown in FIG. 1, it is feasible to extract one of the following hydroxycinnamic acid derivative triterpene alcohol ester from rice bran: cycloartenyl ferulate, 24-methylene cycloartenyl ferulate, campesteryl ferulate and β-sitosteryl ferulate.

### Embodiment 2

[Synthesis of hydroxycinnamic acid derivative triterpene alcohol ester] Hydroxycinnamic acid derivative triterpene alcohol ester can be synthesized according to the method in the above-mentioned non-patent literature 1. Firstly, 5 g trans-ferulic acid and 5 g anhydrous propionic acid (Aldrich) are dissolved in 15 ml pyridine (Aldrich) to be agitated for 48 h in nitrogen, and then the resulting 1.2 g 4-propionyl ferulic acid and 400 mg cholesterol (SIGMA) are dissolved in 100 ml dichloromethane, to which 900 mg 2-chloro-1,3-dimethylimidazolinium (DMC; Aldrich) is added.

Thereafter, 200 mg pyridine is slowly added while the resulting solution is thoroughly cooled. After agitation for 4 h under room temperature, 1000 ml distilled water is added and the dichloromethane layer is washed clean first with 1000 ml dilute hydrochloric acid and then with saturated aqueous solution of sodium bicarbonate. The lower layer is saved and dehydrated with anhydrous sodium sulfate, from which the solvent is then removed in an evaporator. The resulting solid dry substance is dissolved in chloroform, and the sample thereof is spotted onto a Silica 60 plate to be separated through thin-layer chromatography with chloroform as developing solvent.

While fluorescence is excited through 365 nm UV ray, the spot present in red under 50% sulfate detection is scrapped. Silica is stripped into a mixture solvent of chloroform:methanol (1:1, v/v). The resulting cholesterol 4-propionyl ferulic acid is added into 0.1M potassium hydroxide methanol solution. Cholesterol ferulic ester can be obtained after dehydration at 50° C. for 20 min.

Replacing cholesterol with other triterpene alcohols can achieve synthesis of other hydroxycinnamic acid derivative triterpene alcohol ester.

### Embodiment 3

[Measurement of LPS-stimulated NF-κB activation in RAW264.7 murine macrophage strain] The murine macrophage RAW264.7 is preserved in DMEM containing 10% fetal bovine serum. After treatment in DMEM with 1 μM cycloartenyl ferulate (CAF) for 22 h, it is further stimulated for 2 h in 1 μg/ml lipopolysaccharide (LPS).

The nucleoprotein of RAW264.7 cell is extracted with Transfactor Extraction kit (BD Biosciences, USA), and then is bound with the following NF-κB consensus sequence of biotin-labeling reaction for 60 min in reaction solution consisting 100 mM HEPES (pH 7.6), 5 mM EDTA, 50 mM ammonium sulfate, 5 mM DTT, 150 mM KCl, 1% (v/v) Tween20 and 0.0001% poly (dI-dC) (Amersham Biosciences, USA).

NF-κB consensus sequence:
5′-AGTTGAGGGGACTTTCCCAGGC-3′

Except these experimental groups, control group reacting with 90-fold unlabeled consensus sequence, along with supershift group using anti-p50 polyclonal antibody (Abcam, USA) and anti-p65 polyclonal antibody (Santa Cruz Biotechnology, INC., USA) are established to make comparison.

The conjugation reactant is electrophoretically isolated in a 6.0% polyacrylamide gel containing 1.0% Tris/borate/EDTA buffer solution, and is then transferred onto a nylon membrane (Presoak Pall Biodyne B, Whatman, USA). After transfer, the membrane is heated at 85° C. for 30 min for cross-linking with DNA and then reacts with Streptavidin-labeled peroxidase (SIGMA, USA) for 15 min.

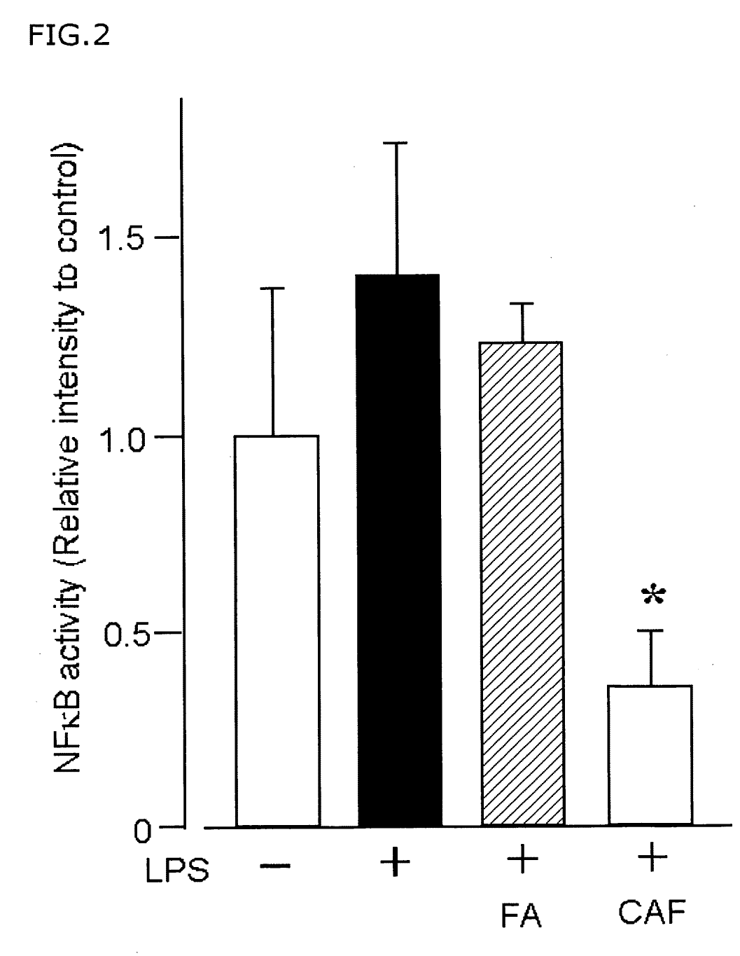

The peroxidase activity is detected with Immobilon Western Chemiluminescent HRP substrate (Millipore, USA) and the luminous intensity is quantified by Image J (National Institutes of Health, USA) as shown in FIG. 2.

In the present embodiment, the NF-κB activation is examined under the presence of cycloartenyl ferulate (1 μM: CAF), as one of hydroxycinnamic acid derivative triterpene alcohol ester, and ferulic acid (1 μM: FA), as in vivo metabolite of CAF. FIG. 2 shows the result.

The result shown in FIG. 2 reveals that RAW264.7 cell may acquire a higher NF-κB activation without stimulation, whereas NF-κB activation shows a rising trend with LPS stimulation. It further reveals that ferulic acid barely inhibits the NF-κB activation, but cycloartenyl ferulate, one of the hydroxycinnamic acid derivative triterpene alcohol ester, markedly inhibits the NF-κB activation.

### Embodiment 4

[Expressions of iNOS and IL-1β mRNA under the LPS stimulation in RAW264.7 murine macrophage strain as interpreted on semi-quantitative RT-PCR] The total RNA is extracted by Sepazol-RNA (Nacalai tesque Inc., Japan) from RAW264.7 cell receiving a variety of stimulations. The RNA amount is quantified on the basis of absorption intensity at 260 nm. The obtained total RNA can harvest cDNA with the help of M-MLV reverse transcriptase (Promega, USA), Oligo (dT) 12-18 primer and a reaction solution consisting of RNase inhibitor. The resulting cDNA is amplified for the target DNA using the below specific primers and Taq DNA polymerase (TaKaRa Japan).

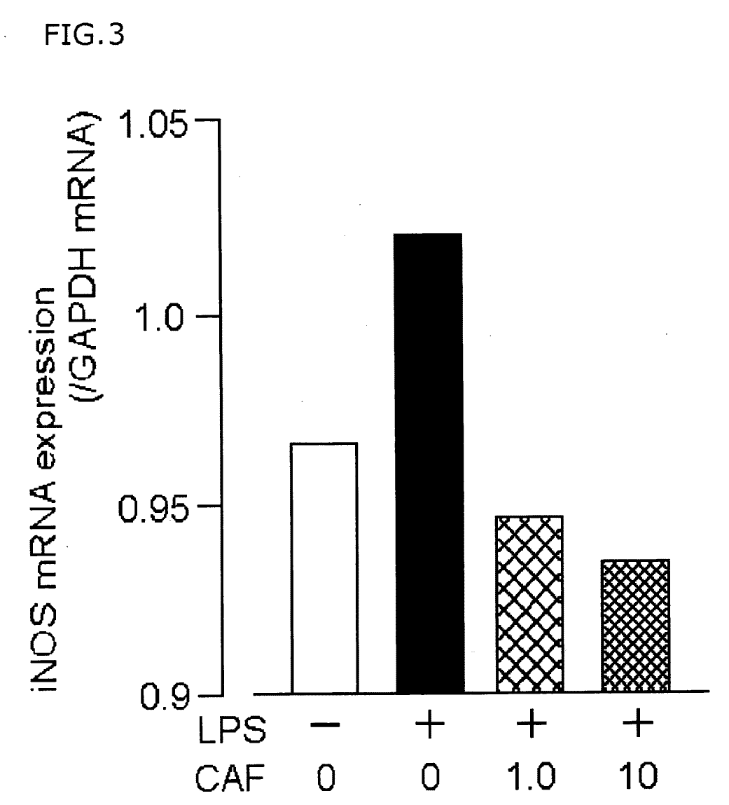

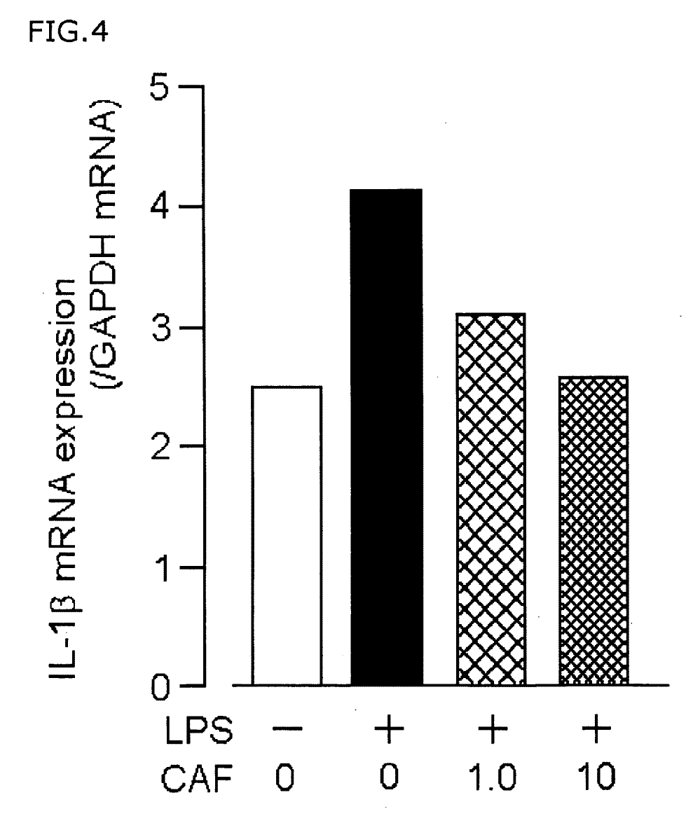

Furthermore, the hot start PCR method is employed. The PCR system (Bio-Rad, Japan) operates as follows: reacting at 94° C. for 5 min, 94° C. for Imin, 55° C. for 1.5 min, 72° C. for Imin, and after repeated cycles, reacting at 72° C. for 5 min. For iNOS, IL-1β and β-actin primer, the fragments of 479, 387, 374 and 349 bp are added respectively. The resulting fragments are separated on a 2% agarose gel electro-phoresis, stained with ethidium bromide and quantified by Image J (National Institutes of Health, USA) on the basis of staining intensity. FIGS. 3 and 4 show the outcome.

iNOS:
forward primer, 5′-GCCTCGCTCTGGAAAGA-3′;
Reverse primer, 5′-TCCATGCAGACAACCTT-3′;
IL-1β:
forward primer, 5′-TGCAGAGTTCCCCAACTGGTACATC-3′
Reverse primer: 5′-GTGCTGCCTAATGTCCCCTTGAATC-3′
β-actin:
forward primer, 5′-TGGAATCCTGTGGCATCCATGAAAC-3′
Reverse primer: 5′-TAAAACGCAGCTCAGTAACAGTCCG-3′

The efficacy of cycloartenyl ferulate (1 μM and 10 μM: CAF), one of the hydroxycinnamic acid derivative triterpene alcohol ester, is characterized as shown in FIGS. 3 and 4.

Based upon the results shown in FIGS. 3 and 4, RAW264.7 cell may acquire a higher expression of iNOS and IL-1β mRNA even without stimulation, which is consistent with the higher level of NF-κB activation with no stimulation. It is also found that the expression of iNOS and IL-1β mRNA in RAW264.7 cell is more up-regulated as a result of LPS stimulation. On the other hand, cycloartenyl ferulate (1 μM and 10 μM: CAF), as one of hydroxycinnamic acid derivative triterpene alcohol ester, can markedly suppress the expression of iNOS and IL-1β mRNA.

### Embodiment 5

[Inflammatory Intestinal Disease Model] Dextran sulfate (DSS: MW36000-50000, MP Biomedicals) is dissolved in water to prepare solutions of 3%, 1% and 0.5%. The C57BL/6J mice (male, 8 weeks) are allowed free drinking. Mice in the 3% DSS dosing group are euthanatized on Day 7 post-dosing and those in the 1% and 0.5% DSS dosing groups on Day 14 post-dosing to extract the diseased colonic specimens.

For the γ-oryzanol group, mixture of hydroxycinnamic acid derivative triterpene alcohol ester, according to embodiment 1, γ-oryzanol is confected into mixture suspension in normal saline containing 0.5% carboxymethyl cellulose and 0.01% Tween 20. Daily 50 mg/kg/day oral dosing is adopted 2 days before DSS free drinking.

Control group: solvent alone, 6 mice

3% DSS group: solvent alone oral dosing+3% DSS free drinking, 5 mice

3% DSS+γ-oryzanol group: γ-oryzanol oral dosing+3% DSS free drinking, 5 mice

1% DSS: solvent alone oral dosing+1% DSS free drinking, 6 mice

1% DSS+γ-oryzanol group: γ-oryzanol oral dosing+1% DSS free drinking, 6 mice

0.5% DSS solvent alone oral dosing+0.5% DSS free drinking, 6 mice

0.5% DSS+γ-oryzanol group: γ-oryzanol oral dosing+0.5% DSS free drinking, 6 mice

1) Disease Activity Index (DAI): From the start of free drinking, body weight measurement, palpation of stool hardness and presence/absence of bloody stool (using occult blood test kit) are interpreted each morning, and Disease Activity Index (DAI) is calculated based on the following stipulations:
(A) Loss in body weight: none (0), 1-5% (1), 5-10% (2), 10-15% (3), >15% (4)
(B) Stool property: normal (0), soft (2), watery (4)
(C) Occult blood kit test: none (0), slightly green (1), green within 3 seconds (2), fresh green upon reaction (3), dark blue upon reaction (4)

{(A)+(B)+(C)}/3=DAI

2) HE staining: Within the 3% DSS dosing group, the colonic lesion specimen (rectum ascending colon) is removed on Day 7 post-DSS dosing and then is fixed with neutral formalin to prepare pathological section through HE-staining.

3) Myeloperoxidase activity (MPO activity): The colonic lesion specimen (rectum˜ascending colon) is removed. After discarding the intestinal content, MPO activity corresponding to the total protein amount in the intestinal lesion specimen is measured as the index of neutrophilic granulocyte infiltration.

1) DAI score: As compared with the control group, there is no notable difference in body weight in either 1% DSS or 0.5% DSS dosing group. Also in the γ-oryzanol dosing group, there is no difference as compared with any control group.

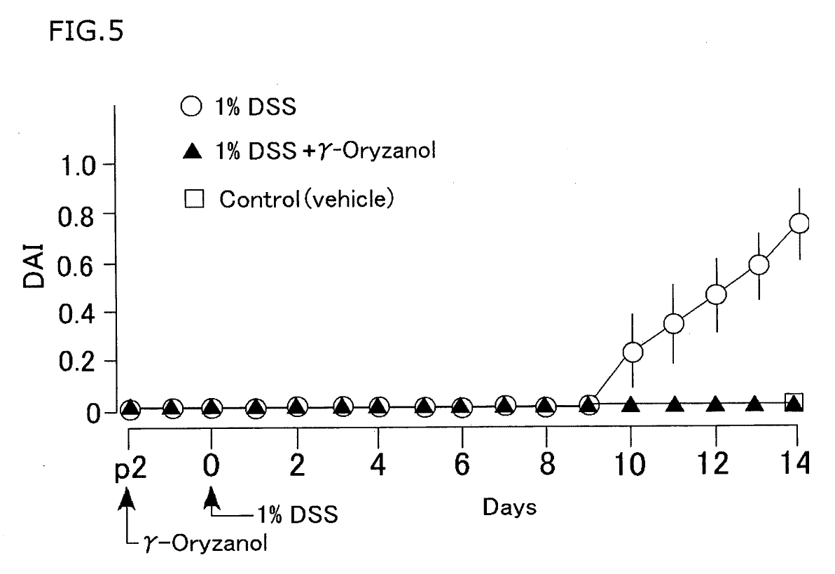

By evaluation method of DAI score, there is no difference between 0.5% DSS dosing group and control group, both with DAI score of zero. On the other hand, in 1% DSS dosing group as shown in FIG. 5, stool softening and mild occult blood come forth as of Day 10 post-DSS dosing and the DAI score prominently rises to 0.72±0.13 on Day 14 post-DSS dosing. The DAI score of each individual in the γ-oryzanol dosing group is zero, proving a remarkable symptomatic improvement.

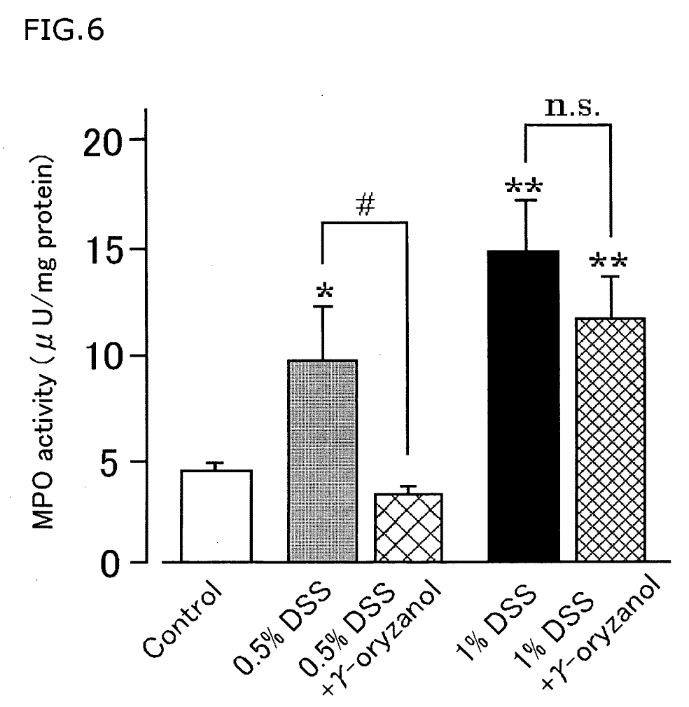

2) MPO activity: As shown in FIG. 6, within the colonic lesion specimen in the 0.5% and 1% DSS dosing groups, the myeloperoxidase activity, as an index of neutrophilic granulocyte infiltration, increases with the rising DSS concentration. Within the γ-oryzanol dosing group, 0.5% DSS dosing almost reduces the MPO activity down to resting level. Despite the inhibitive trend in the 1% DSS dosing group, the difference is not considered to be significant.

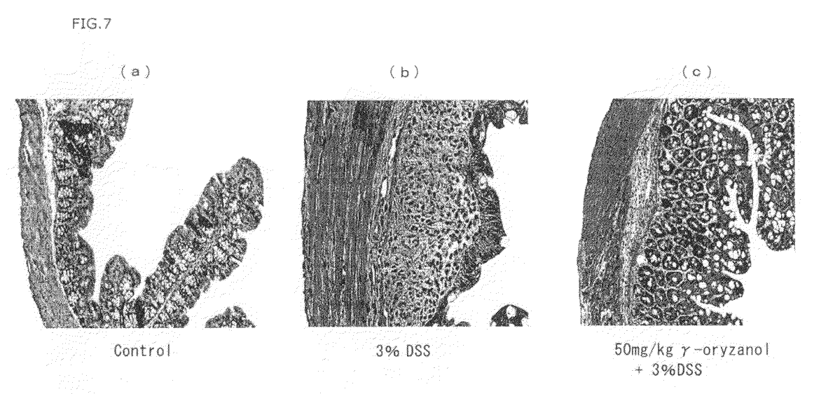

3) HE staining: As shown in FIG. 7, (b) depicts the colonic lesion in Week 1 of 3% DSS dosing, wherein slough-off of mucous epithelium, infiltration and hypertrophy of inflammatory cells into submucous tissues, and partial hypertrophy of intima can be observed. On the other hand, as shown in (c), the γ-oryzanol dosing group reveals the infiltration of inflammatory cells into submucous tissues whereas the hypertrophies of submucous tissues and intima lessened markedly, and slough-off of mucous epithelium is milder compared with the DSS dosing group. (a) acts as the control group.

### Embodiment 6

Murine 3T3-L1 precursor adipocytes (IFO50416, HSRRB) are purchased to be cell cultured in Dulbecco's Modified Eagle Medium (DMEM) (Nissui Pharmaceutical) containing 10% FBS (SIGMA) employing 24-well plate. The culture medium is replaced every 48 h to carry out proliferation and the cell culture is processed into flasks of 25 cm² and 75 cm². The cultured cell is collected and placed in a BICELL freezer (JRAIA) to cool down to −85° C. at a rate of 1° C./min before stored at −85° C.

[Differentiation to adipocytes] The murine 3T3-L1 precursor adipocytes in 75 cm² flask are treated with trypsin EDTA (Immuno-Biological Laboratories) to be collected into a 50 ml plastic tube and the supernatant thereof is removed after centrifugal rotation at 1000 rpm for 5 min. The cells are subject to suspending with fresh culture medium added, and the resulting cell suspension is loaded apart into a 6-well plate and cultured into confluence. After the confluence of cells, the culture medium is substituted by a culture medium for induction and differentiation with 5 μg/ml insulin (WAKO PURE CHEMICAL INDUSTRIES), 0.5 mmol/l 3-isobutyl-1-methyl-xanthine (IBMX) (SIGMA) and 1 μmol/l dexamethasone (WAKO PURE CHEMICAL INDUSTRIES). The substituting culture medium is replaced freshly every 48 h for 7 consecutive days, which is replaced thereafter by a proliferation medium to culture cells for 2 more days.

[Effect upon adiponectin in adipocytes] 100 μl experimental culture medium containing respectively 1 μM γ-oryzanol, 1 μM β-sitosterol, 1 μM trans-ferulic acid, 1 μM cholesterol and 1 μM troglitazone is added apart. The culture medium containing troglitazone is adopted for positive control whereas the culture medium containing DMSO only for negative control. After treatment for 22 h and removal of the culture medium, NF-κB is activated after 2 h incubation in a culture medium containing 1 μg/ml LPS, 50 ng/ml recombinant TNF-α and 100 U/ml recombinant IFN-γ. The culture medium of NF-κB activation is removed 2 h later, and each culture medium is collected 24 h after the start of experimental medium treatment. The collected culture medium is mixed with SDS-PAGE sample buffer, and the mixture is subjected to SDS-PAGE and Western blotting after 3 min boiling.

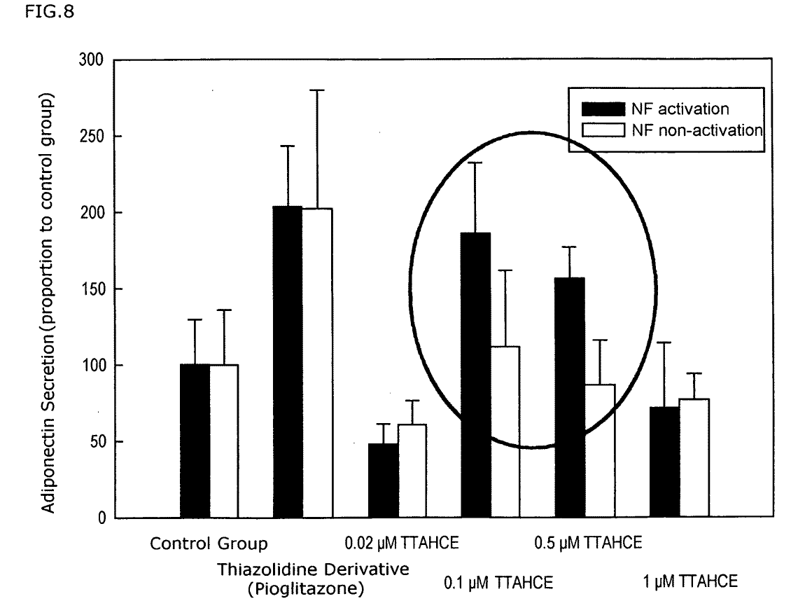

In addition, the ABC reaction taking advantage of the conjugation capacity of biotin and avidin is employed for detection. More specifically, Biotinylated Anti-mouse Adiponectin polyclonal antibody (R&D system, Inc) firstly reacts with a primary antibody, then with ABC solution (WAKO PURE CHEMICAL INDUSTRIES) containing complex of horseradish peroxidase labelled biotin and avidin, and is finally detected with Chemiluminescent HRP Substrate (MILLIPORE) through chemiluminescence method or Sigma FAST DAB tablet. The detection bands are read by a digital flat-bed scanner and then subjected to image analysis by Image-J to measure the adiponectin level to obtain result shown in FIG. 8.

Based upon the result as shown in FIG. 8, within the murine adipocytes with LPS-activated NF-κB, hydroxycinnamic acid derivative triterpene alcohol ester (TTAHCE) in moderate concentration can strongly promote the secretion of adiponectin. To put it another way, hydroxycinnamic acid derivative triterpene alcohol ester inhibits the NF-κB activation and promotes the secretion of adiponectin as antagonizing factor of type-2 diabetes.

1. A NF-κB activation inhibitor, comprising a compound represented by chemical structural formula depicted in the following Chemical Formula 1 [wherein R₁ represents a hydroxy group; R₂ represents a hydroxy group, a methoxy group or an alkoxy group; and R₃ represents a triterpene] or a salt thereof as active ingredient.

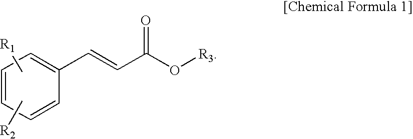

2. The NF-κB activation inhibitor according to claim 1, which comprises a hydroxycinnamic acid derivative triterpene alcohol ester or a salt thereof as active ingredient.

3. The NF-κB activation inhibitor according to claim 1, which comprises a cycloartenyl ferulate or a salt thereof as active ingredient.

4. The NF-κB activation inhibitor according to claim 1, which comprises a β-sitosteryl ferulate or a salt thereof as active ingredient.

5. The NF-κB activation inhibitor according to claim 1, which comprises a stigmasteryl ferulate or a salt thereof as active ingredient.

6. The NF-κB activation inhibitor according to claim 1, which comprises a 24-methylene cycloartenyl ferulate or a salt thereof as active ingredient.

7. The NF-κB activation inhibitor according to claim 1, which comprises a campesteryl ferulate or a salt thereof as active ingredient.

8. The NF-κB activation inhibitor according to claim 2, which comprises a cycloartenyl ferulate or a salt thereof as active ingredient.

9. The NF-κB activation inhibitor according to claim 2, which comprises a β-sitosteryl ferulate or a salt thereof as active ingredient.

10. The NF-κB activation inhibitor according to claim 2, which comprises a stigmasteryl ferulate or a salt thereof as active ingredient.

11. The NF-κB activation inhibitor according to claim 2, which comprises a 24-methylene cycloartenyl ferulate or a salt thereof as active ingredient.

12. The NF-κB activation inhibitor according to claim 2, which comprises a campesteryl ferulate or a salt thereof as active ingredient.
